(19) United States
(12) Patent Application Publication
(10) Pub. No.: US 2023/0181159 A1
(43) Pub. Date: Jun. 15, 2023

(54) Ultrasound Imaging System with Tactile Probe Control

(71) Applicant: GE Precision Healthcare LLC, Wauwatosa, WI (US)

(72) Inventors: Andreas Kremsl, St. Wolfgang i.S. (AT); Christian Perrey, Mondsee (AT)

(21) Appl. No.: 17/547,409

(22) Filed: Dec. 10, 2021

(51) Int. Cl. A61B 8/00

(52) U.S. Cl. CPC A61B 8/467; A61B 8/54; A61B 8/4254; A61B 8/4472; A61B 8/461

(57) ABSTRACT

An ultrasound imaging probe includes a housing, a circuit board assembly disposed within the housing including a microcontroller, and a motion sensor operably connected to the microcontroller and capable of sensing motion of the housing based on at least one operator interaction with the housing to generate sensor data, a memory chip disposed within the housing and storing information regarding particular operational configurations for the ultrasound imaging probe associated with stored sensor data representing types of stored operator interactions, and a power source disposed at least partially within the housing and operably connected to the assembly. The microcontroller is configured to receive sensor data from the motion sensor in response to the operator interaction with the housing, to compare the received sensor data with the stored sensor data, and to change an operational configuration of the ultrasound probe in response to matching the received sensor data with stored sensor data.

### BACKGROUND OF THE INVENTION

Embodiments of the present disclosure relate generally to an ultrasound imaging system including one or more ultrasound probes and, more particularly, to function control systems for the ultrasound imaging system and the ultrasound imaging probes.

Various medical conditions affect internal organs and bodily structures. Efficient diagnosis and treatment of these conditions typically require a physician to directly observe a patient's internal organs and structures. On many occasions, imaging using an ultrasound imaging system is utilized to obtain images of a patient's internal organs and structures in a minimally invasive manner. The ultrasound images can be obtained utilizing a probe that is located either externally or internally relative to the patient.

By way of example, ultrasound images for non-interventional procedures, such as those obtained for transthoracic echocardiography (TTE), can be obtained by placing the probe against the exterior of the chest of the patient when operating the ultrasound imaging system. Alternatively, ultrasound images for interventional procedures, such as for transesophageal echocardiography (TEE) and/or intracardiac echocardiography (ICE), are obtained by inserting the probe within the body of the patient, e.g., into the esophagus, while the ultrasound imaging system is in operation.

Ultrasound procedures are typically performed in examination, interventional and operating room (open heart surgery) situations where imaging of internal structures of the patient is required. The ultrasound imaging system utilized in performing the ultrasound procedure typically includes the probe, a processing unit, and a monitor. The probe is connected to the processing unit which in turn is connected to the monitor. In operation, the processing unit sends a triggering signal to the probe. The probe then emits ultrasonic signals via an imaging element within the probe into the patient. The probe then detects echoes of the previously emitted ultrasonic signals. Then, the probe sends the detected signals to the processing unit which converts the signals into images. The images are then displayed on the monitor.

Typically, during the operation of the ultrasound imaging system, control of the operational state of the ultrasound imaging system, including, but not limited to the parameters for the operation of the ultrasound probe and/or the presentation of the ultrasound images obtained via the probe on the monitor, is selected utilizing various control devices disposed on one or both of the monitor and the probe. The control devices enable the operator to select the desired operation for the ultrasound imaging system and/or the probe, such as to switch on power to the probe, to select a scan mode, and to freeze an ultrasound image being presented on the monitor, among others. These control devices take the form of various buttons, knobs, rotatable wheels, switches and the like that are located on the monitor (such as in the form of graphic user interfaces (GUIs)) and/or on the ultrasound probe. The control devices are accessed directly by the operator to change the operational state of the ultrasound imaging system and ultrasound probe as desired to achieve the desired images for presentation on the monitor.

However, while the control devices enable the operator to select the desired operational condition for the ultrasound system and/or ultrasound probe, the placement of the control devices on each of the monitor and the probe can present certain issues with regard to doing so. More specifically, in order to select a particular configuration or operational setup for the ultrasound imaging system, the selection process involves a complex sequence of control device selection on one or both of the monitor and the probe. This results in a longer than desired amount of time required to achieve the selected operational configuration/setup for the ultrasound imaging system, particularly in situations where the probe and monitor are spaced apart and the operator has to move between the monitor and location where the probe is being utilized to select the necessary control devices.

Further, when an ultrasound imaging system is used to assist an operator in performing a medical procedure, the operator may hold an ultrasound probe in one hand, while holding a medical instrument in their other hand. This may make it difficult for the operator to adjust settings on the ultrasound imaging system because both of the operator's hands are busy positioning devices. Unfortunately, an additional operator may be needed to assist in adjusting operating settings of the ultrasound imaging system during such a procedure, or to hold the ultrasound probe, or to perform other tasks due to the inability to access controls while holding the probe and another medical instrument.

To address this issue, some prior art ultrasound probes include various control devices thereon that enable the operator to change the operational settings of the ultrasound imaging system using only the hand holing the ultrasound probe, such as those disclosed in US Patent Application Publication No. US2012/0197131, entitled Probe Mounted Ultrasound System Control Interface, the entirety of which is expressly incorporated herein by reference for all purposes. However, even with these mechanical rocker control switches disposed directly on the ultrasound probe, the operator must closely control the operation of the control device in order to select the desired operation configuration for the ultrasound imaging system, which still takes the operator's focus off of the position and control of the probe and other medical instrument.

In an attempt to overcome this issue, other ultrasound probes have been developed that include touchpads on the housing of the probe, as disclosed in U.S. Pat. No. 8,827,909, entitled Ultrasound Probe, the entirety of which is expressly incorporated herein by reference for all purposes. The operator can move their fingers contacting the probe housing in sliding manners along the touchpads to provide control signals to the ultrasound imaging system that alter the operation of the probe.

However, while the touchpads enable alterations of the operational configuration of the ultrasound imaging system to be made directly on the ultrasound probe by moving the fingers of the operator along areas of the probe housing, the nature of the touchpads make any fine control and/or discernment of the movements of the operator's fingers difficult to quantify an correlate to particular control signals.

Therefore, it is desirable to develop a system and method for the control of the setup and switching of the operational configuration for the ultrasound imaging system that avoids the requirement for complex control device sequences to achieve the desired configuration. Further, it is desirable to provide a system that enables an operator to perform a medical procedure using an ultrasound probe in one hand and a medical instrument in another hand without an additional operator may decrease the number of operators necessary for the procedure, improve controllability of the process, and render it more intuitive.

### BRIEF DESCRIPTION OF THE DISCLOSURE

In one exemplary embodiment of the invention, an ultrasound imaging probe includes a housing, an integrated circuit disposed within the housing, the integrated circuit comprising a microcontroller, and at least one motion sensor operably connected to the microcontroller and capable of sensing motion of the housing based on at least one operator interaction with the housing to generate sensor data representing the at least one operator interaction, a memory chip disposed within the housing and operably connected to the microcontroller, the memory chip storing information regarding particular operational configurations for the ultrasound imaging probe associated with stored sensor data representing types of stored operator interactions, and a power source disposed at least partially within the housing and operably connected to the integrated circuit, wherein the microcontroller is configured to receive sensor data from the at least one motion sensor in response to at least one operator interaction with the housing, to compare the received sensor data with the stored sensor data, and to change an operational configuration of the ultrasound imaging probe in response to matching the received sensor data with stored sensor data.

In another exemplary embodiment of the invention, an ultrasound imaging system includes a processing unit configured to receive and process acquired ultrasound image data to create ultrasound images derived from the ultrasound image data, a display operably connected to the processing unit to present the created ultrasound images to a user, a memory unit operably connected to the processing unit, and an ultrasound imaging probe operably connected to the processing unit to obtain the ultrasound image data, the ultrasound imaging probe comprising a housing, a transducer element disposed within the housing to obtain and send the ultrasound image data to the processing unit, an integrated circuit disposed within the housing, the integrated circuit comprising a microcontroller, and at least one motion sensor operably connected to the microcontroller and capable of sensing motion of the housing based on at least one operator interaction with the housing to generate sensor data representing the at least one operator interaction, a memory chip disposed within the housing and operably connected to the microcontroller, and a power source disposed at least partially within the housing and operably connected to the integrated circuit, wherein at least one of the memory unit and the memory chip contains information regarding particular operational configurations for the ultrasound imaging probe and ultrasound imaging system associated with stored sensor data representing types of stored operator interactions, and wherein at least one of the processing unit and the microcontroller is configured to receive sensor data from the at least one motion sensor in response to at least one operator interaction with the housing, to compare the received sensor data with the stored sensor data, and to change an operational configuration of at least one of the ultrasound imaging system or the ultrasound imaging probe in response to matching the received sensor data with stored sensor data.

In a further exemplary embodiment of the invention, a method of controlling the operational configuration of an ultrasound imaging system and an ultrasound imaging probe includes the steps of providing an ultrasound imaging system comprising a processing unit configured to receive and process acquired ultrasound image data to create ultrasound images derived from the ultrasound image data, a display operably connected to the processing unit to present the created ultrasound images to a user, a memory unit operably connected to the processing unit, and an ultrasound imaging probe operably connected to the processing unit to obtain the ultrasound image data, the ultrasound imaging probe comprising a housing a transducer element disposed within the housing to obtain and send the ultrasound image data to the processing unit, an integrated circuit disposed within the housing, the integrated circuit comprising a microcontroller, and at least one motion sensor operably connected to the microcontroller and capable of sensing motion of the housing based on at least one operator interaction with the housing to generate sensor data representing the at least one operator interaction, a memory chip disposed within the housing and operably connected to the microcontroller, and a power source disposed at least partially within the housing and operably connected to the integrated circuit, wherein at least one of the memory unit and the memory chip contains information regarding particular operational configurations for the ultrasound imaging probe and ultrasound imaging system associated with stored sensor data representing types of stored operator interactions, and wherein at least one of the processing unit and the microcontroller is configured to receive sensor data from the at least one motion sensor in response to at least one operator interaction with the housing, to compare the received sensor data with the stored sensor data, and to change an operational configuration of at least one of the ultrasound imaging system or the ultrasound imaging probe in response to matching the received sensor data with stored sensor data, sensing at least one operator interaction with the probe housing via the at least one motion sensor, receiving sensor data for the at least one operator interaction, comparing the received sensor data with stored sensor data associated with an operational configuration change, and changing the operational configuration of at least one of the ultrasound imaging system or the ultrasound imaging probe in response to the sensed at least one operator interaction with the housing.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 is a schematic view of an ultrasound imaging system according to an embodiment of the disclosure.

FIG. 2 is a schematic view of the ultrasound imaging system of FIG. 1 with an active ultrasound probe.

FIG. 3 is an isometric view of an ultrasound probe according to an exemplary embodiment of the disclosure.

FIG. 4 is a schematic view of a printed circuit board (PCB) and power source disposed within the probe of FIG. 3.

FIG. 5 is a schematic view of one surface of the PCB of FIG. 4.

FIG. 6 is a schematic view of the other surface of the PCB of FIG. 4.

FIG. 7 is a flowchart illustrating a method of altering the operational configuration of the ultrasound imaging system of FIG. 1 according to an exemplary embodiment of the disclosure.

### DETAILED DESCRIPTION

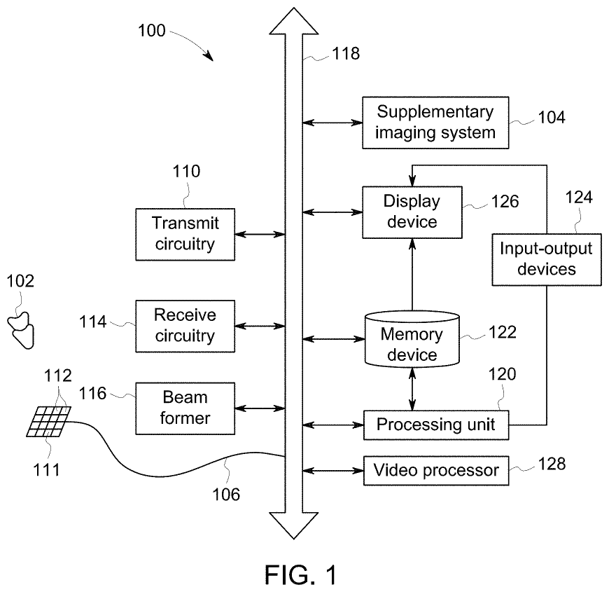

FIG. 1 illustrates an exemplary ultrasound imaging system 100 for optimal visualization of a target structure 102 for use during ultrasound imaging procedures. For discussion purposes, the system 100 is described with reference to a non-interventional ultrasound imaging probe, such as a TTE probe, utilized with the system 100. However, in certain embodiments, other types of imaging probes may be employed with the imaging system 100, such as interventional ultrasound probes, including a TEE probe, or an ICE probe, among others.

In one embodiment, the ultrasound imaging system 100 employs ultrasound signals to acquire image data corresponding to the target structure 102 in a subject. Moreover, the ultrasound imaging system 100 may combine the acquired image data corresponding to the target structure 102, for example the cardiac region, with supplementary image data. The supplementary image data, for example, may include previously acquired images and/or real-time intra-operative image data generated by a supplementary imaging system 104 such as a CT, MRI, PET, ultrasound, fluoroscopy, electrophysiology, and/or X-ray system. Specifically, a combination of the acquired image data, and/or supplementary image data may allow for generation of a composite image that provides a greater volume of medical information for use in accurate guidance for an interventional procedure and/or for providing more accurate anatomical measurements.

Accordingly, in one embodiment shown in FIG. 1, the ultrasound imaging system 100 includes a probe 106 such as an interventional or non-interventional ultrasound probe. The probe 106 is adapted for external use, i.e., the probe 106 is placed on the skin of the patient to image internal structures of the patient, or the probe 106 can be configured to be operated in a confined medical or surgical environment such as a body cavity, orifice, or chamber corresponding to an object or subject, e.g., the patient.

To that end, in certain embodiments the ultrasound imaging system 100 includes transmit circuitry 110 that may be configured to generate a pulsed waveform to operate or drive one or more transducer elements 111 or a transducer array 112, as controlled by the user via the system 100, or a separate control device or handle (not shown) as part of the system 100. The transducer elements 111/array 112 are configured to transmit and/or receive ultrasound energy and may comprise any material that is adapted to convert a signal into acoustic energy and/or convert acoustic energy into a signal. For example, the transducer elements 111 may be formed from a piezoelectric material, such as lead zirconate titanate (PZT), or a capacitive micromachined ultrasound transducer (CMUT) according to exemplary embodiments. The probe 106 may include more than one transducer element 111, such as two or more transducer elements 111 optionally arranged in a matrix transducer array 112, or separated from each other on the probe 106. The transducer elements 111 produce echoes that return to the transducer elements 111/array 112 and are received by receive circuitry 114 for further processing. The receive circuitry 114 may be operatively coupled to a beamformer 116 that may be configured to process the received echoes and output corresponding radio frequency (RF) signals.

Further, the system 100 includes a processing unit 120 communicatively coupled to the beamformer 116, the probe 106, and/or the receive circuitry 114, over a wired or wireless communications network 118. The processing unit 120 may be configured to receive and process the acquired image data, for example, the RF signals according to a plurality of selectable ultrasound imaging modes in near real-time and/or offline mode.

Moreover, in one embodiment, the processing unit 120 may be configured to store the acquired volumetric images, the imaging parameters, and/or viewing parameters in a memory device 122. The memory device 122, for example, may include storage devices such as a random access memory, a read only memory, a disc drive, solid-state memory device, and/or a flash memory. Additionally, the processing unit 120 may display the volumetric images and or information derived from the image to a user, such as a cardiologist, for further assessment on a operably connected display 126 for manipulation using one or more connected input-output devices 124 for communicating information and/or receiving commands and inputs from the user, or for processing by a video processor 128 that may be connected and configured to perform one or more functions of the processing unit 120. For example, the video processor 128 may be configured to digitize the received echoes and output a resulting digital video stream on the display device 126.

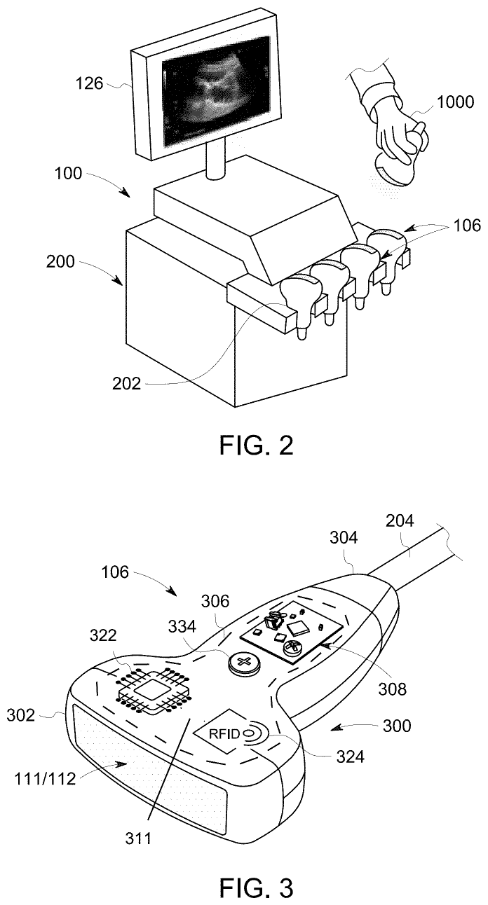

Looking now at the exemplary illustrated embodiment of FIGS. 2 and 3, ultrasound imaging system 100 includes a base unit 200 within which the processing unit 120, memory device 122, and video processor 128 are located. The base unit 200 also supports the display/monitor 126 which can additionally be formed as a touch screen to function as the input-output device 124. The base unit 200 additionally includes a number of docking stations 202 thereon within which the probes 106, such as a transthoracic echocardiography (TTE) probe, can be supported on the base unit 200. The probes 106 are connected to the base unit 200, and thus the processing unit 120, via a cord or wire 204 (FIG. 3) but in the illustrated exemplary embodiment of FIG. 2 the probes 106 are each selectively wirelessly connected to the imaging system 100/base unit 200, with the cord 204 being selectively detachable from the probe 106. In addition, for the probes 106, the docking stations 202 can provide a recharging function to a rechargeable power source or battery (not shown) disposed within the probe 106, either through the cord 204 or wirelessly, e.g., by using an induction charging process in a known manner.

Looking now at FIGS. 3-6, the probe 106, of whatever type, is formed with a housing 300 which encloses the functional components of the probe 106. The housing 300 includes a forward end 302 that is adapted to be placed against or within the object/patient to be imaged and which houses the transducer elements 111/array 112, and a rearward end 304 to which the cord 204 (if present) is attached. Between the forward end 302 and the rearward end 304 the housing 300 forms a handle 306 that can be readily grasped with one hand 1000 (FIG. 2) in order to manipulate and position the forward end 302 of the probe 106 to obtain the desired ultrasound images.

Within the housing 300, as illustrated by the dotted line in FIG. 3 indicating a portion of the housing 300 that is removed for illustration purposes only, the probe 106 includes one or more sensor boards 308 that provide control signals to operate the transducer elements 111/array 112 under the control of the operator. The one or more sensor boards 308 are operably connected to the processing unit 120 and that contain one or more sensors 310 thereon. Then sensors 310 are capable of sensing various actions taken by the operator with regard to the housing 300 in order to control the operation of the imaging system 100 and/or the probe 106. While in the illustrated exemplary embodiment the sensors 310 are shown as being located directly on the sensor boards 308, in alternative embodiments the sensors 310 can be disposed on other portions of the housing 300, such as on an interior surface 311 of the housing 300 or in other locations within the housing 300 other than on the sensor boards 308.

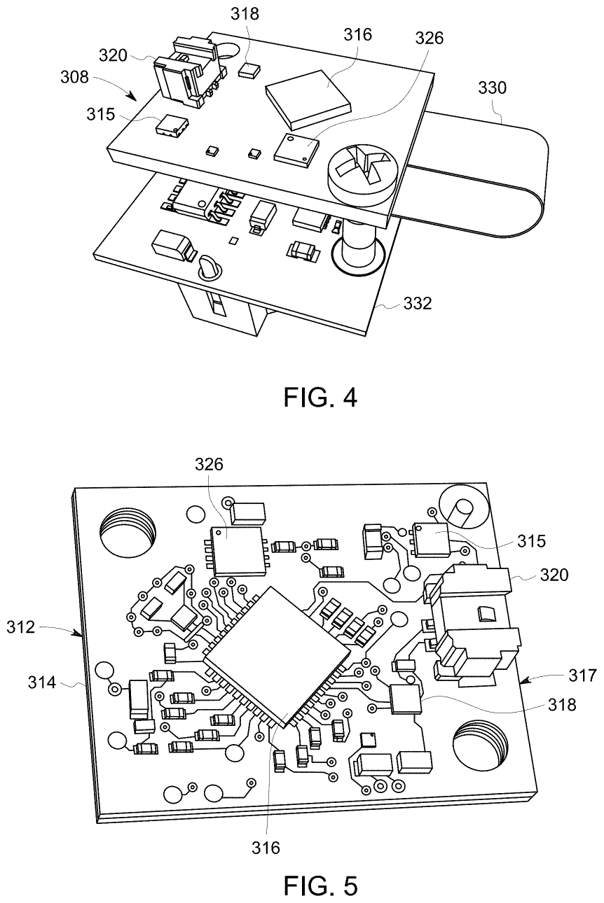

Looking now at FIGS. 4-6, in the illustrated exemplary embodiment the one or more sensor boards 308 are each formed as an integrated circuit/printed circuit board assembly (PCBA) 312. The PCBA 312 includes a main board 314 including the sensors 310 mounted thereto. The sensors 310 disposed on the main board 314 are formed as any suitable type of motion sensor 310, including but not limited to, a drop sensor, an accelerometer, a pressure sensor, a capacitance sensor, and/or a gyroscopic sensor, or combinations thereof, among others. The PCBA 312 can also include sensors 310 other than motion sensors, such as a temperature sensor, a light sensor 315, and/or a humidity sensor, among others. The motion sensors 310 disposed on the main board 314 are capable of sensing actions performed on the housing 300 by the operator and using the PCBA 312 and/or processing unit 120 to interpret the actions into controls regarding the operation of the imaging system 100 and/or probe 106.

The PCBA 312 additionally includes a microcontroller 316 disposed on the main board 314 and which is operably connected to each of the sensors 310. The signals received or sensed by each of the sensors 310 are directed to the microcontroller 316 which can function to interpret the signal/sensor data to affect the operation of the imaging system 100 and/or probe 106, and/or which can configure the signal/sensor data for transmission to the processing unit 120. In addition, the microcontroller 316 can be operably connected to the transmit circuitry 110, the receive circuitry 114 and the beamformer 116 in order to affect the operation of the probe 106 in response to signals/sensor data from the sensors 310 and/or from the processing unit 120.

To send the signals/sensor data to the processing unit 120, in embodiments where a cord 204 connects the housing 300 and the base unit 200, the signals/sensor data can be directly transmitted along internal wiring of the cord 204 operably connected between the PCBA 312 and the processing unit 120. Alternatively, as illustrated in the exemplary embodiments of FIGS. 3-6, the housing 300 and/or PCBA 312 can include a wireless communication module 317 to wirelessly connect the PCBA 312 to the base unit 200/processing unit 120. In the illustrated exemplary embodiment of FIGS. 3-5, the wireless module 317 is a radio frequency identification (RFID) transponder 318 that operates using any suitable wireless protocol, e.g., 802.11g, WiFi, Bluetooth®, Zigbee®, BLE, WLAN, etc. The transponder 318 can be formed either separately from the PCBA 312 as an RFID module 324 that is connected to the PCBA 312, or as a part of the PCBA 312 and is operably connected to the microcontroller 316. The transponder 318 can send wireless signals to the processing unit 120 containing the signals/sensor data received from the sensors 310, and can receive signals from the processing unit 120 containing control instructions for the operation of the probe 106. The transponder 318 also is connected to an antenna 320 in order to boost signals to and from the transponder 318 for proper reception and transmission of the signals.

As shown in the exemplary embodiment of FIG. 4, the housing 300 can also contain a memory chip 322 that is operably connected to the PCBA 312. The memory chip 322 can contain information regarding operational configurations for the operation of the probe 106 associated with various signals/sensor data 310 obtained by the sensors 310 and transmitted to the microcontroller 316. Using the memory chip 322, the microcontroller 316 can compare the signals/sensor data to the stored configurations present on the memory chip 322 to determine of the sensor data corresponds to a stored operational configuration associated with that sensor data. The data stored on the memory chip 322 can alternatively be stored within memory unit 122 and accessed by the processing unit 120 to compare sensor data from the PCBA 312 to locate any operational configurations for the probe 106 associated with the sensor data. In addition, to operational configurations for the probe 106, the operational configurations stored in one or both of the memory unit 122 and memory chip 322 can include operational configurations for the imaging system 100 and/or the probe 106.

To ensure that the signals sent from and received by the PCBA 312 are authorized by the imaging system 100, as show in the exemplary embodiment of FIG. 5, the PCBA 312 can include an authentication module/chip 326 on the main board 314 and connected to the microcontroller 316. The authentication chip 326 can block unauthorized control, data or other signals from being sent or received by the PCBA 312 to prevent any unauthorized alterations to the operational configurations of the probe 106 and/or the imaging system 100. To accomplish this, the authentication chip 326 add a component to any signal sent from the PCBA 312 to the imaging system 100/processing unit 120 that provides the necessary authentication for acceptance of the signal by the imaging system 100/processing unit 120. Conversely, the authentication chip 326 can decode and/or recognize a similar signal component disposed within a signal sent to the PCBA 312 to authenticate the signal as coming from the imaging system 100/processing unit 120.

To power the PCBA 312 and the sensors 310, microcontroller 316, RFID transponder 318 and other components on the main board 314, as illustrated in the exemplary embodiment of FIGS. 3, 4 and 6, the PCBA 312 includes a power supply connector 328 on the main board 314 that is connected via suitable wiring 330 to a power supply board 332. The power supply board 332 is operably engaged by a power source, such as a power wire (not shown) disposed within the cord 204 (if present) or a battery 334 disposed within the housing 300. Electrical power supplied from the power source, i.e., the power wire or the battery 334, passes through the power supply board 332 which conditions the electrical power for proper usage by the PCBA 312 and the components thereon. As stated previously, the battery 334 can be a rechargeable battery 334 that is recharged by a docking station 202 on the base unit 200 using either the power wire in the cord 204, or using a wireless recharging process, as is known.

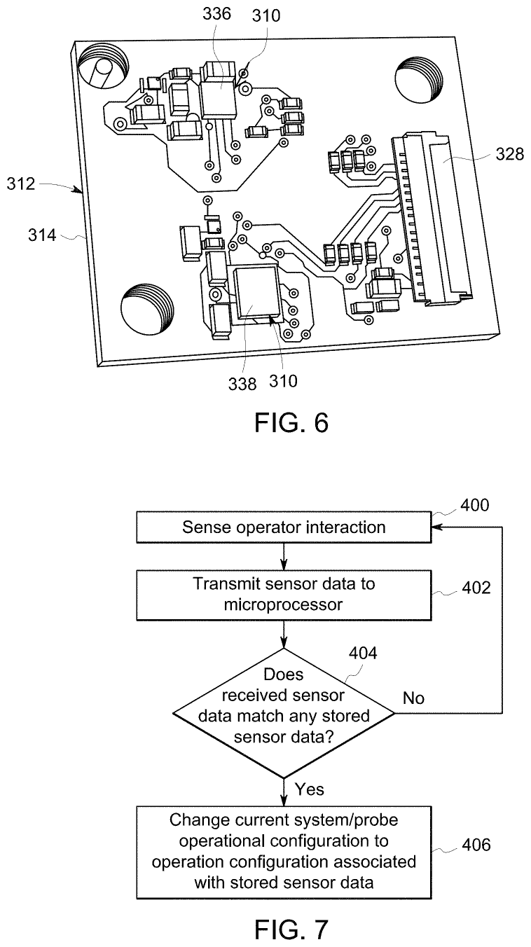

As the PCBA 312 includes one or more motion sensors 310, these sensors 310 can provide sensor data to the microcontroller 316 regarding the type(s), strength and duration of motion applied to the probe 106 including the PCBA 312. More specifically, in the exemplary illustrated embodiment of FIGS. 5 and 6, the PCBA 312 includes each of an accelerometer 336 and a gyroscope or gyro sensor 338. As such, the accelerometer 336 can sense any contact with the housing 300 that creates a vibration in the housing 300, while the gyro sensor 338 can sense the angular motion and/or velocity of the housing 300. In this manner, various types of contact and/or motion of the housing 300 of the probe 106 can be sensed by the accelerometer 336 and/or gyro sensor 338 in order to provide control instructions to the microprocessor 316/processing unit 120/imaging system 100 concerning an operational configuration for the imaging system 100 and/or the probe 106.

The motion sensors 310, and in particular the accelerometer 336 and/or gyro sensor 338 sense/record operator commands via the interaction of the operator directly with the housing 300 and handle 306 for the probe 106 to control the operation of the ultrasound imaging system 100 based on these commands. Due to the ability of the accelerometer 336 and the gyro sensor 338 to sense small vibrations or angular rotations of the probe housing 300/handle 306, the commands can be performed by the operator through taps of a finger on the housing 300, which are sensed by the accelerometer 336, or movements of the housing 300, which are sensed by the gyro sensor 338.

Each command for a particular operational configuration of the imaging system and/or probe 106 is associated with a predetermined type, number and/or pattern of interactions performed by the operator on the housing 300. As stated previously, the list of the operational configurations for the imaging system 100 and/or probe 106 are stored in one or both of the memory unit 122 and/or memory chip 322 in association with the stored sensor data/operator interaction that identify the particular command to change to the selected operational configuration. Thus, as shown in block 400 of the method of FIG. 7, initially an operator interaction with the housing 300/handle 306 of the probe 106 is sensed by the motion sensor 310, i.e., either the accelerometer 336 and/or the gyro sensor 338. In block 402, motion sensor 310 transmits the sensor data to the processing unit 120 and/or the microprocessor 316 for processing, such as into a format corresponding to the stored sensor data representing the stored operator interactions associated with the operator commands in the memory unit 122/memory chip 322.

In decision block 404, the processing unit 120/microprocessor 316 compares the received sensor data with the stored data associated with the operator commands to determine if the received sensor data matches the stored data associated with a particular operator command. If no, the processing unit 120/microprocessor 316 discards the received sensor data as relating to an inadvertent operator interaction and returns to block 400 to await additional sensor data. However, if the received sensor data does correspond to a stored sensor data/operator interaction, in block 406 the processing unit 120/microprocessor 316 accesses the information relating to the operator command associated with the stored sensor data/operator interaction and initiates a change in the operational configuration of one or both of the imaging system 100 and/or the probe 106 according to the stored information for the operator command. After completing the operational configuration change, the processing unit 102/microprocessor 316 returns to block 400 to await receipt of additional sensor data.

With regard to the types of operator interactions the operator can have with the housing 300/handle 306 that are associated with particular operator commands/sensor data, according to an exemplary embodiment, each interaction is able to be performed exclusively by the single hand utilized by the operator to hold the probe housing 300/handle 306 in order to enable the operator to select the desired command using only the hand already positioned on the probe housing 300/handle 306. For example, the operator interactions that can be sensed by the motion sensors 310, e.g., the accelerometer 336 and/or the gyro sensor 338, and translated into sensor data/operator commands can be performed by:
1) tapping/making a finger tap gesture or action with one or more fingers on or against the probe 106/housing 300/handle 306;
2) shaking the probe 106/housing 300/handle 306;
3) waving the probe 106/housing 300/handle 306 in a particular direction or directions;
4) rotating the probe 106/housing 300/handle 306 in a particular direction; and/or
5) inverting the probe 106/housing 300/handle 306, such as for a specified time period.

In addition, the operator commands/sensor data from the operator interactions can result in different changes to the operational configuration for the imaging system 100/probe 106 depending on the current operational configuration for the imaging system 100/probe 106 when the operator command is made. Alternatively described, a single operator interaction can be associated with a first change to the operational configuration of the imaging system 100 and/or probe 106 when the imaging system 100 and/or probe 106 is in a first state, and can be associated with a different, second change to the operational configuration of the imaging system 100 and/or probe 106 when the imaging system 100 and/or probe 106 is in a second state. For example, a single finger tap/tap gesture or action can:
1) trigger the probe 106 into an active configuration if the probe 106 is currently inactive;
2) freeze/pause the current image obtained by the probe 106 and being presented on the display 126 if the probe 106 is currently actively scanning;
3) unfreeze/unpauses the probe 106 currently in freeze/pause mode; or
4) change a preset setting for an active probe currently in scan mode, where the probe is not in contact with the object or patient being imaged.

Further, besides the operator interaction being defined as a single action taken relative to the probe 106/housing 300/handle 306, a single tap, pre-defined tap gesture or action sequences may be utilized. For example, the operator interaction associated with an operator command can be a simple or complex sequence of interactions with the probe 106/housing 300/handle 306. From the simple to the complex, these sequences of interactions can be a number of finger taps on the probe 106/housing 300/handle 306, a sequence of finger taps with pauses between some or all of the finger taps, similar to a Morse code sequence, and a combination of finger tap gestures or actions and other motions of the probe 106/housing 300/handle 306, such as waving, shaking, and/or rotating the probe 106/housing 300/handle 306.

In addition, the imaging system 100 and/or the probe 106 can come with a number of operator interactions pre-defined for certain operator commands, but this list can optionally be modified by changing certain operator interaction/operator command associations, and/or by adding additional operator interactions/operator command associations to the memory unit 122/memory chip 322 to customize the control of the imaging system 100 and/or probe 106 capable by the operator interactions/operator command associations.

Further, as opposed to an operator interaction being associated with a single operator command, an operator interaction can initiate a single event or a series of actions by the imaging system 100 or the probe 106. For example:
1) when an operator taps 2 times on the probe 106/housing 300/handle 306, the imaging system 100/probe 106 increases image depth;
2) when an operator taps 3 times on the probe 106/housing 300/handle 306, the imaging system 100/probe 106 decreases image depth;
3) while scanning, when an operator taps 2 times on the probe 106/housing 300/handle 306, the imaging system 100/probe 106 freezes and stores cine to archive;
4) when an operator taps 2 times slow on inactive probe 106/housing 300/handle 306, the imaging system 100/probe 106 is activated with preset A;
5) when an operator taps 2 times fast on inactive probe 106/housing 300/handle 306, the imaging system 100/probe 106 is activated with preset B.

In an alternative embodiment, the probe 106/housing 300/handle 306 can include colored areas (not shown) indicating where tapping should be performed on the probe 106/housing 300/handle 306 in order to enable the motion sensors 310 to most effectively register the operator interactions by tapping. Further, the probe 106/housing 300/handle 306 can include a light source (not shown) or other visual indicator that provide the operator with confirmation that the operator interaction has or has not been recognized as an operator command by the imaging system 100/probe 106.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An ultrasound imaging probe comprising:
a housing;
a microcontroller disposed within the housing;
at least one motion sensor disposed within the housing and operably connected to the microcontroller, the at least one motion sensor and capable of sensing motion of the housing based on at least one operator interaction with the housing to generate sensor data representing the at least one operator interaction;
a memory chip disposed within the housing and operably connected to the microcontroller, the memory chip storing information regarding particular operational configurations for the ultrasound imaging probe associated with stored sensor data representing types of stored operator interactions; and
a power source disposed at least partially within the housing and operably connected to the circuit board assembly,
wherein the microcontroller is configured to receive sensor data from the at least one motion sensor in response to at least one operator interaction with the housing, to compare the received sensor data with the stored sensor data, and to change an operational configuration of the ultrasound imaging probe in response to matching the received sensor data with stored sensor data.

2. The ultrasound imaging probe of claim 1, wherein the at least one motion sensor is at least one of an accelerometer, a pressure sensor, a capacitance sensor, a gyroscopic sensor, or combinations thereof.

3. The ultrasound imaging probe of claim 2, wherein the at least one operator interaction is a finger tap against the housing.

4. The ultrasound imaging probe of claim 3, wherein the at least one operator interaction is a pre-determined sequence of finger taps against the housing.

5. The ultrasound imaging probe of claim 2, wherein the at least one operator interaction is selected from the group consisting of: shaking the housing, waving the housing, rotating the housing, inverting the housing, and combinations thereof.

6. The ultrasound imaging probe of claim 5, wherein the at least one operator interaction comprises:
at least one finger tap against the housing; and
at least one of shaking the housing, waving the housing, rotating the housing, inverting the housing, and combinations thereof.

7. The ultrasound imaging probe of claim 1, wherein the stored operator interaction is associated with multiple operational configurations for the ultrasound imaging probe, with each operational configuration associated with a different current operating state for the ultrasound imaging probe.

8. The ultrasound imaging probe of claim 1, wherein the stored operator interaction is associated with a first change to the operational configuration of the ultrasound imaging probe when the ultrasound imaging probe is in a first current operating state, and is associated with a second change to the operational configuration of the ultrasound imaging probe when the ultrasound imaging system probe is in a second current operating state.

9. The ultrasound imaging probe of claim 7, wherein the stored operator interaction:
triggers the probe into an active operating state if the probe is currently in an inactive operating state;
triggers the probe into a paused scanning operating state if the probe is in an active scanning operating state; and
triggers the probe into an active scanning operating state if the probe is in a paused scanning operating state.

10. The ultrasound imaging probe of claim 1, further comprising a wireless module operably connected to the microcontroller.

11. An ultrasound imaging system comprising:
a processing unit configured to receive and process acquired ultrasound image data to create ultrasound images derived from the ultrasound image data;
a display operably connected to the processing unit to present the created ultrasound images to a user;
a memory unit operably connected to the processing unit; and
an ultrasound imaging probe operably connected to the processing unit to obtain the ultrasound image data, the ultrasound imaging probe comprising:
a housing;
a transducer element disposed within the housing to obtain and send the ultrasound image data to the processing unit;
a microcontroller disposed within the housing;
at least one motion sensor disposed within the housing and operably connected to the microcontroller, the at least one motion sensor capable of sensing motion of the housing based on at least one operator interaction with the housing to generate sensor data representing the at least one operator interaction;
a memory chip disposed within the housing and operably connected to the microcontroller; and
a power source disposed at least partially within the housing and operably connected to the circuit board assembly,
wherein at least one of the memory unit and the memory chip contains information regarding particular operational configurations for the ultrasound imaging probe and ultrasound imaging system associated with stored sensor data representing types of stored operator interactions, and wherein at least one of the processing unit and the microcontroller is configured to receive sensor data from the at least one motion sensor in response to at least one operator interaction with the housing, to compare the received sensor data with the stored sensor data, and to change an operational configuration of at least one of the ultrasound imaging system or the ultrasound imaging probe in response to matching the received sensor data with stored sensor data.

12. The ultrasound imaging system of claim 11, wherein the at least one motion sensor is at least one of an accelerometer, a pressure sensor, a capacitance sensor, a gyroscopic sensor, or combinations thereof.

13. The ultrasound imaging probe of claim 11, wherein the at least one operator interaction is selected from the group consisting of: at least one finger tap against the housing, shaking the housing, waving the housing, rotating the housing, inverting the housing, and combinations thereof.

14. The ultrasound imaging probe of claim 11, wherein the stored operator interaction is associated with multiple operational configurations for the at least one of the ultrasound imaging system or the ultrasound imaging probe, with each operational configuration associated with a different current operational configuration for the at least one of the ultrasound imaging system or the ultrasound imaging probe.

15. The ultrasound imaging probe of claim 11, wherein the stored operator interaction is associated with a first change to the operational configuration of the at least one of the ultrasound imaging system or the ultrasound imaging probe when the at least one of the ultrasound imaging system or the ultrasound imaging probe is in a first current operating state, and is associated with a second change to the operational configuration of the at least one of the ultrasound imaging system or the ultrasound imaging probe when the at least one of the ultrasound imaging system or the ultrasound imaging probe is in a second current operating state.

16. The ultrasound imaging probe of claim 11, further comprising a wireless module operably connected to the microcontroller.

17. A method of controlling the operational configuration of an ultrasound imaging system and an ultrasound imaging probe, the method comprising the steps of:
providing an ultrasound imaging system comprising:
a processing unit configured to receive and process acquired ultrasound image data to create ultrasound images derived from the ultrasound image data;
a display operably connected to the processing unit to present the created ultrasound images to a user;
a memory unit operably connected to the processing unit; and
an ultrasound imaging probe operably connected to the processing unit to obtain the ultrasound image data, the ultrasound imaging probe comprising:
a housing;
a transducer element disposed within the housing to obtain and send the ultrasound image data to the processing unit;
a circuit board assembly disposed within the housing, the circuit board assembly comprising:
a microcontroller; and
at least one motion sensor operably connected to the microcontroller and capable of sensing motion of the housing based on at least one operator interaction with the housing to generate sensor data representing the at least one operator interaction;
a memory chip disposed within the housing and operably connected to the microcontroller; and
a power source disposed at least partially within the housing and operably connected to the circuit board assembly,
wherein at least one of the memory unit and the memory chip contains information regarding particular operational configurations for the ultrasound imaging probe and ultrasound imaging system associated with stored sensor data representing types of stored operator interactions, and wherein at least one of the processing unit and the microcontroller is configured to receive sensor data from the at least one motion sensor in response to at least one operator interaction with the housing, to compare the received sensor data with the stored sensor data, and to change an operational configuration of at least one of the ultrasound imaging system or the ultrasound imaging probe in response to matching the received sensor data with stored sensor data;
sensing at least one operator interaction with the probe housing via the at least one motion sensor;
receiving sensor data for the at least one operator interaction;
comparing the received sensor data with stored sensor data associated with an operational configuration change; and
changing the operational configuration of at least one of the ultrasound imaging system or the ultrasound imaging probe in response to the sensed at least one operator interaction with the housing.

18. The method of claim 17, wherein the step of comparing the received sensor data with the stored sensor data further comprises the step of determining a current operational state of at least one of the ultrasound imaging system or the ultrasound imaging probe.

19. The method of claim 18, wherein the step of changing the operational configuration of at least one of the ultrasound imaging system or the ultrasound imaging probe comprises:
performing a first change to the operational configuration of the at least one of the ultrasound imaging system or the ultrasound imaging probe when the at least one of the ultrasound imaging system or the ultrasound imaging probe is in a first current operational configuration state; and
performing a second change to the operational configuration of the at least one of the ultrasound imaging system or the ultrasound imaging probe when the at least one of the ultrasound imaging system or the ultrasound imaging probe is in a second current operating state.

20. The method of claim 17, wherein the step of sensing at least one operator interaction with the probe housing comprises sensing a sequence of operator interactions with the probe housing.
